(19) United States
(12) Patent Application Publication
(10) Pub. No.: US 2024/0041760 A1
(43) Pub. Date: Feb. 8, 2024

(54) VACCINATION FOR PROTECTING POULTRY AGAINST A POULTRY PATHOGEN

(71) Applicant: Intervet Inc., Rahway, NJ (US)

(72) Inventors: Willem Pieter Cornelis Pulskens, Germert (NL); Paul Vermeij, St. Anthonis (NL); Theodorus Jansen, Venray (NL); Wilhelmus Gerardus Johannes Degen, Doetinchem (NL); Carla Christina Schrier, Boxmeer (NL)

(73) Assignee: Intervet Inc., Rahway, NJ (US)

(21) Appl. No.: 18/264,446

(22) PCT Filed: Feb. 7, 2021
(86) PCT No.: PCT/EP2022/052811

§ 371 Date: Aug. 7, 2023

(30) Foreign Application Priority Data
Feb. 8, 2021 (EP) 21155675.8

(51) Int. Cl. A61K 9/00; A61K 39/17; A61K 39/112; A61K 39/15; A61P 31/14; A61P 31/04

(52) U.S. Cl. CPC A61K 9/006; A61K 39/17; A61K 39/0275; A61K 39/15; A61P 31/14; A61P 31/04; A61K 2039/541

(57) ABSTRACT

The invention pertains to a vaccine comprising non-live antigen of a poultry pathogen and a mucoadhesive adjuvant, for use in boosting an immune response in a poultry animal directed against the poultry pathogen by administering the vaccine mucosally to the poultry animal. The invention also pertains to a vaccine comprising a liquid pharmaceutically acceptable carrier, a non-live antigen of a poultry pathogen and a mucoadhesive adjuvant, as well as a method of boosting an immune response in a poultry animal, which immune response is directed against a poultry pathogen, by administering a vaccine mucosally to the poultry animal, the vaccine comprising a non-live antigen of the said poultry pathogen and a mucoadhesive adjuvant.

### GENERAL FIELD OF THE INVENTION

The invention in general pertains to methods for protecting poultry against poultry pathogens by vaccination, and vaccines suitable for applying in those methods.

### BACKGROUND OF THE INVENTION

Poultry animals (also “poultry”) are kept as a source of animal protein throughout the world. Moreover, poultry are able to adapt to most geographical areas and conditions, they are not expensive to buy, they have rapid generation time and a high rate of productivity, and they do not require large areas of land. Poultry production systems differ, ranging from rural farming to highly industrialized and vertically integrated systems. Backyard poultry production is distributed in most rural and peri-urban areas of the world, and is mainly based on the rearing of domestic poultry, both terrestrial and aquatic. Intensive poultry production is most common in developed countries, but in the last few decades, many developing countries have also adopted this system in order to meet the increasing demand for animal proteins. In recent times, the risk of transmission of certain transboundary poultry diseases to previously unaffected areas has increased as a result of globalization and the possible persistence and spread of infectious disease agents (poultry pathogens) through domestic and wild reservoirs. The widespread distribution of Newcastle disease (ND), Infectious Bursal Disease (IBD) and avian influenza (AI) that have occurred over the last twenty years provide examples of the negative impact of such diseases on the poultry producing sector and on society as a whole. Different strategies can be implemented to effectively prevent and control the spread of animal diseases at international, national and farm levels and poultry disease control plans often include the use of vaccination. Vaccines are, in fact, an important component of poultry disease prevention and control worldwide. Their use in poultry production is traditionally aimed at avoiding or minimising the emergence of clinical disease at farm level and thus increasing production. Vaccines and vaccination programmes vary widely, depending on several local factors (e.g. type of production, level of biosecurity, local pattern of disease, status of maternal immunity, vaccines available, costs and potential losses). Although poultry vaccination is generally managed by the poultry industry, it has only rarely been applied in the framework of a disease eradication programme at national or regional level to control a few major poultry diseases (e.g. Al and ND). The first expected outcome of the administration of a poultry vaccine is that birds will develop immunity to pathogens and thus be protected against disease. The results that may be achieved through the use of vaccination can be different. For example protection against the clinical form of the disease, reduction of susceptibility to infection (a higher infectious dose is required to trigger infection in vaccinated birds than in those unvaccinated) and reduction of infectivity (e.g. shedding) in case of infection.

The use of vaccines for the control of poultry diseases vaccination is generally tailored and adjusted according to local factors that may influence the strategy, the design and the effectiveness of the vaccination programme once it has been implemented. Several different factors should be taken into account, including the type of poultry production (e.g. commercial or rural), the organisation of the industry (e.g. vertical integration), the densities of different bird species, the prevailing disease situation, vaccine availability, the use of other vaccines, the prevalence of other diseases, the resources available (e.g. manpower and equipment) and also the costs involved. Many methods and types of vaccines are available for poultry vaccination such as in ovo vaccination, spray vaccination, SC (subcutaneous) and IM (intramuscular) vaccination, wing-web vaccination and administration via the drinking water. All kinds of vaccines can be applied ranging from subunits to live vaccines. Parenteral vaccination using inactivated vaccines, typically via intramuscular injection is often applied and typically very effective, but expensive to perform since each animal has to be handled and treated separately. Mass vaccination via spray, aimed at reaching the mucosal surfaces of the eyes and/or respiratory tract and/or gastrointestinal tract is convenient and inexpensive to perform but generally requires the antigen to be live, which negatively affects safety.

### OBJECT OF THE INVENTION

It is an object of the invention to devise a new poultry vaccination strategy that is safe, effective and easy to perform, in particular suitable for mass-vaccination.

### SUMMARY OF THE INVENTION

In order to meet the object of the invention a vaccination method has been devised wherein a vaccine comprising non-live antigen of a poultry pathogen and a mucoadhesive adjuvant, is used for boosting an (existing) immune response in a poultry animal, the (existing) immune response being directed against the poultry pathogen, by administering the vaccine mucosally to the poultry animal.

In the art it is commonly recognised that inducing an adequate immune response in a poultry animal using a non-live antigen, can only be accomplished by parenteral administration. Although mucosal administration is preferred from a convenience and economical point of view, by applying a non-live antigen to a mucosal surface, typically will not lead to an adequate immune response directed against a poultry pathogen and thus, will not lead to adequate protection against that pathogen. The reason for this is not quite clear but indeed, the mucosal immune system is complex and not yet fully understood in both human and animal health. Mucosal (immune) sites are constantly exposed to foreign particles from the environment and are adapted to the presence of these particles. Therefore, the mucosal immune system is believed to favour tolerance over an immune response. This makes it more difficult to induce an immune response, in particular when using a non-live (e.g. inactivated) antigen.

However, applicant found that mucosal administration may very well be effective for boosting an immune response that is already present in the animal, such that adequate protection against the pathogen can be arrived at. For this, it appears to be essential that the vaccine as used comprises next to the non-live antigen, a so called mucoadhesive adjuvant. This adjuvant is used as delivery system and carrier of the antigen to improve the adhesion hereof to mucosal membranes, thereby enhancing local mucosal residence time and controlled local release of the antigen. Apparently, by adjusting the vaccine formulation with addition of an adjuvant that has mucoadhesive characteristics, local retention time is enhanced, thereby improving the antigen uptake and subsequent exposure to the underlying immune system and reducing the efflux by flow or local degradation by proteases.

For a range of several poultry pathogens it was found that this way an immune response could be boosted to a level that approached boosting by a second parenteral vaccination shot. It has thus been shown that by using this type of administration, at least as a second immunization, a very good immune response can be obtained using safe (non-live) antigen and an easy administration route. Mucosal administration namely can be accomplished by mass vaccination, e.g. using a spray vaccinator, as known in the art. The prime immune response can be the result of any type of immunization as known to be effective for poultry such as in ovo vaccination, spray vaccination of day-old chicks with a live vaccine, parenteral vaccination, wild-type infection, or even mucosal vaccination with the present vaccine. The gist of the invention is that the booster vaccination can be accomplished effectively via mucosal vaccination, and thus via mass vaccination if desired, using a non-live antigen, as long as a mucoadhesive adjuvant is present in the vaccine to carry and deliver the antigen. This way, an immune response can be boosted, and as commonly known for poultry animals, provide protection against a poultry pathogen, i.e. prevent, mitigate or cure the actual infection or a disease resulting form that infection.

The invention also pertains to a vaccine comprising a liquid pharmaceutically acceptable carrier, a non-live antigen of a poultry pathogen and a mucoadhesive adjuvant, as well as to a method of boosting an immune response in a poultry animal, which immune response is directed against a poultry pathogen, by administering a vaccine mucosally to the poultry animal, the vaccine comprising a non-live antigen of the said poultry pathogen and a mucoadhesive adjuvant.

### Definitions

A vaccine is a constitution that is safe to administer to a host, and protects this host against a post vaccination infection with a pathogen (a pathogenic micro-organism such as a virus or bacterium), i.e. a constitution that prevents or reduces this infection by the pathogen, or prevents or reduces a clinical disease that results from the infection, typically by interfering with the pathogen itself, for example via antibodies, in the vaccinated host. Vaccination thus prevents, or at least diminishes, the level of infection and/or prevents, or at least diminishes, the level of clinical disease resulting from that infection. Typically, a vaccine comprises an antigen in a pharmaceutically acceptable carrier such as an aqueous, often buffered, solution, or any other liquid carrier.

A non-live antigen of a pathogen is any substance or compound, other than the live pathogen as such (either in wild-type or attenuated form), against which an immunological response is to be elicited, such that the corresponding virulent pathogen or one or more of its virulence factors will be recognized by the host's immune system as a result of this immune response, and are ultimately at least partly neutralized. Typical examples of non-live antigen of a pathogen are inactivated wild type pathogen (such a killed virus or bacterium), subunits of the pathogen such as surface expressed proteins and toxins. The latter two may or may not be recombinantly expressed. With regard to poultry pathogens, many non-live antigens are commonly known in the art.

An adjuvant is an agent that is able to non-specifically stimulate an immune response. In principal, each agent that is able to favor or amplify a particular process in the cascade of immunological events, ultimately leading to a better immunological response (i.e. the integrated bodily response to an antigen, in particular one mediated by lymphocytes and typically involving recognition of antigens by specific antibodies or previously sensitized lymphocytes), can be defined as an adjuvant. An adjuvant is in principle not required for the said particular immunological process to occur, but favors or amplifies the said process.

A mucoadhesive adjuvant is a hydrophilic polymer (e.g. having hydroxyl, carboxyl, amine or sulphate groups), having a molecular weight of at least 2,000 (two thousand) Da, preferably at least 5,000 (five thousand) Da, which is to be used as delivery system and carrier of vaccine antigens to improve the adhesion to mucosal membranes enhancing local mucosal residence time and controlled local release of the antigen. Preferably the polymer has a pK value that differs from the pH of the target mucosa of the host animal to be vaccinated, such that ionization of the polymer upon contact with the mucosal surface is induced. The pH of poultry mucosal surfaces of the eye and oral cavity are typically near neutral, between 6.5 and 7.5, for healthy animals. Preferably the mucosal adjuvant is a biodegradable polymer, i.e. a polymer that is capable of being decomposed by the target organism.

Factors influencing mucoadhesion are the hydrophilicity, molecular weight and pK value. Mucoadhesive polymers possess numerous hydrophilic functional groups, such as hydroxyl and carboxyl. These groups allow hydrogen bonding with the substrate, swelling in aqueous media, thereby allowing maximal exposure of potential anchor sites. In addition, swollen polymers have the maximum distance between their chains leading to increased chain flexibility and efficient penetration of the substrate. Regarding molecular weight, the interpenetration of polymer molecules is favored by low-molecular-weight polymers, whereas entanglements are favored at higher molecular weights. The optimum molecular weight for the maximum mucoadhesion depends on the type of polymer, with adhesive forces increasing with the molecular weight of the polymer up to 100,000-300,000 Da. Beyond this level, there is no further gain. Regarding the pK value, the pH at the mucoadhesive to mucosal surface interface can influence the adhesion of mucoadhesives possessing ionizable groups. Many mucoadhesives used in drug delivery are polyanions possessing carboxylic acid functionalities. For an acidic mucoadhesive polymer, if the local pH is above or the pKa of the polymer, it will be largely ionized; if the pH is below the pKa of the polymer, it will be largely unionized. For example, the approximate pKa for the poly(acrylic acid) family of polymers is between 4 and 5. The maximum adhesive strength of these polymers is observed around pH 4-5 and decreases gradually above a pH of 6. The concentration of the mucoadhesive adjuvant is not deemed critical, although in general there will be an optimum for each mucoadhesive adjuvant between 1 and 30% w/w in the final formulation, mostly between 1 and 15% w/w.

Depending on the host animal and target mucosal surface, the following substances are typically used as mucoadhesive adjuvants: natural polymers (or derivates thereof) such as albumin, pectin, cellulose, gelatin, alginate (a series of polymers of various combi's of G-C blocks; final weight varies from 60-600 kDa), chitosan (a series of chitosan polymers ranging from 110-360 kDa), hyaluronic acid; synthetic polymers such as poloxamer (polyalkylene oxide block copolymer; commercially available as Synperonic F127 from Croda Health Care, a 12 kDa ethylene oxide/propylene oxide block copolymer), PEG (polyethylene glycol), PAA (polyacrylic acid, carbomer, Carbopol®, 70-4000 kDa), PLGA (poly (D,L-lactic-co-glycolic acid), PLA (poly D,L-lactic acid), PVP (polyvinylpyrrolidone) and PVA (polyvinylalcohol).

A poultry animal is an animal of a domesticated avian species that can be raised for eggs (layers), meat (broilers) and/or feathers. The term “poultry animal” covers a wide range of birds, from indigenous and commercial breeds of chickens to Muscovy ducks, mallard ducks, turkeys, guinea fowl, geese, quail, pigeons, ostriches and pheasants.

Administering a composition mucosally to an animal means that the composition is provided to come in contact with one or more mucosal surfaces of the animal, such as for example the mucosal surfaces of the eyes, oral, or nasal cavities.

An immune response directed against a pathogen is a response that is able to prevent, mitigate or cure an infection with that pathogen, and/or a disease resulting from that infection.

### FURTHER EMBODIMENTS OF THE INVENTION

In another embodiment of the vaccine for mucosal administration, the immune response in the poultry animal is a result of a natural infection with the poultry pathogen or the administration of a previous vaccine directed against the pathogen. Preferably, the immune response in the poultry animal is a result of a previous vaccination directed against the pathogen, by administering a previous vaccine parenterally or mucosally. For example, the immune response in the poultry animal is a result of a previous vaccination by administering a previous vaccine comprising a non-live antigen of the poultry pathogen parenterally or by administering a previous vaccine comprising a live or non-live antigen of the poultry pathogen mucosally. In an embodiment, the vaccine comprising a non-live antigen of a poultry pathogen and a mucoadhesive adjuvant is administered within 2-6 weeks after administration of the previous vaccine, for example within 2, 3, 4, 5 or 6 weeks.

In yet another embodiment the vaccine is administered ocular (OC) and/or intranasally (IN). These mucosal surfaces have shown to be ideally suitable for mucosal vaccination in the present invention.

In still another embodiment the vaccine is administered by spraying the vaccine on the poultry animal. Spraying may lead to direct administration or indirect administration (after initial spraying onto the animals, e.g. via pecking, breathing etc.) in the eye or nasal cavity.

In yet another embodiment the mucoadhesive adjuvant has a molecular weight above 10,000 Da. Longer polymer chains are believed to be better for adhering to the mucosal surface and sustained delivery of the antigen. A practical upper limit of the molecular weight 1,000,000 (one million) Da, although not much enhancement of the adherence and sustained delivery is expected for any weight above 300,000 Da.

In again another embodiment the mucoadhesive adjuvant has a pKa value below 6. An acidic mucoadhesive adjuvant is less irritant for mucosal surfaces of poultry and the pKa below 6 makes sure there is at least a significant ionisation which improves adherence to the surface. Preferably the mucoadhesive adjuvant has a pKa value between 3 and 5.

In yet again another embodiment the mucoadhesive adjuvant is chosen from an alginate (i.e. a salt of alginic acid) and a polyalkylene oxide blockcopolymer.

In embodiments of the vaccine, the mucoadhesive adjuvant is present in an amount between 1 and 10% w/w, although higher amounts are not excluded. Amounts above 10% however are not preferred for practical (e.g. increased viscosity) and economical (e.g. cost price) reasons.

In an embodiment of the invention the poultry pathogen is a viral pathogen. Viral pathogens are abundant in flocks of poultry that are not easily vaccinated parenterally and therefore the current invention is ideally suitable to combat viral poultry pathogens, such as Newcastle Disease Virus (NDV), Avia Reo Virus (ARV) and Infectious bursal disease virus (IBDV) but also bacterial pathogens such as Salmonella typhimurium.

All the above embodiments also pertain to the vaccine according to the invention and to any methods for treating a poultry animal.

The invention will now be further exemplified using the following specific examples.

### Example 1

Object of the Study

Newcastle disease (ND) is a highly contagious viral disease of poultry that causes economical losses worldwide. In the field, the majority of birds are vaccinated against this virus by live priming followed by a booster vaccination. Mass spray application for the live primo vaccination is commonly applied. The booster vaccination is commonly applied via the intramuscular route. An inactivated antigen in a mucosal, preferably a spray, method for the booster vaccination would be most convenient for the farmer to apply to the flock.

This study aimed at establishing an effective vaccination regime with a live prime vaccine followed by a booster vaccine comprising of an inactivated antigen that can be applied via the mucosal route, for example via a spray application. In particular the efficacy of spray vaccination needs to be assessed, since the useful antigen fraction applied by spray with an actual impact is highly influenced through losses by settlement, drift and evaporation. Therefore, different vaccine formulations comprising of inactivated antigen combined with a mucoadhesive adjuvant were tested. The model antigen that was used in this study is NDV Clone 30, as NDV is a respiratory viral disease for which mucosal (in particular spray) vaccination would be of interest.

Materials and Methods In this study 7 groups (n=13/group) with 1-day old SPF chickens were used. In addition, at the start of the experiment at T=0, an additional 5 hatch mates were used for obtaining reference samples. At T=0 weeks post vaccination (wkpv) all groups received a primo vaccination with live NDV clone 30 (0.1 ml via ocular route). At T=6 wkpv the animals from groups 2-7 received a booster vaccination. Group 2 served as control in which the animals received the booster via an 0.5 ml intramuscular (i.m.) injection at a full dose (“100%,”) of killed (“inac”) NDV with GNE adjuvant (MSD Animal Health). Group 3 received a booster formulation applied via the oculo-nasal route (40% inac NDV with 5% w/v Synperonic F127 (poloxamer 407; Croda health Care) mucoadhesiveve adjuvant; 0.2 ml/animal). To reduce potential impact of loss by spray application (evaporation, settlement) groups 4-7 received the same antigen input per booster formulation but blended within different dosage volumes.

Animals from groups 4 and 5 received different booster formulations applied via fine spray with a total volume of 1 mL per animal. Animals from group 6 and 7 received 2 mL per animal. Groups and treatment are indicated in Table 1.

TABLE 1
Vaccination scheme

| Group | Prime vaccination | Booster vaccination |
|---|---|---|
| 1 | Live NDV | — |
| 2 | Live NDV | Killed NDV, GNE adjuvant, IM, |
| | | 0.5 ml |
| 3 | Live NDV | Killed NDV, 5% Synperonic F127, |
| | | OC/IN, 0.2 ml |
| 4 | Live NDV | Killed NDV, 10% Synperonic F127, |
| | | spray, 1 ml |
| 5 | Live NDV | Killed NDV, 5% Synperonic F127, |
| | | spray, 1 ml |
| 6 | Live NDV | Killled NDV, 10% Synperonic F127, |
| | | spray, 2 ml |
| 7 | Live NDV | Killed NDV, 5% Synperonic F127, |
| | | spray, 2 ml |

T=2 wkpb, T=4 wkpb blood was withdrawn from all animals. To determine serology, sera samples were analyzed for the presence of anti-NDV hemagglutination inhibiting antibodies. For the characterization of peripheral blood mononuclear cells (PBMC), the cells were isolated using Sepmate™ (STEMCELL Technologies) 15 mL tubes and counted using Neubauer counting chamber. CD4 and CD8a cells were assessed using APC and Pacific Blue fluorochromes of Southern Biotech.

Results

The serology results are indicated here below in table 2. As can be seen, the 2 log antibody titers for group 1 remained at a low level of about 5, whereas the antibody level for the boosted groups rose to a very high level of about 12 for the IM group, as expected. However, also the groups boosted with the killed vaccine applied mucosally each showed a significant boosting effect to levels of about 7-8, corresponding to levels for adequate protection against NDV.

TABLE 2
2log Antibody titers 0, 2 and 4 weeks post booster vaccination

| Group | At booster | 2 wpb | 4wpb |
|---|---|---|---|
| 1 | 6 | 5 | 5 |
| 2 | 5 | 12 | 12 |
| 3 | 6 | 8 | 7 |
| 4 | 5 | 7 | 7 |
| 5 | 6 | 8 | 7 |
| 6 | 6 | 7 | 7 |
| 7 | 5 | 8 | 8 |

In table 3 the percentages of 004 and CD8a cells of the total cell population are provided. This is another indication that by applying the vaccine of the invention, a significant boosting effect of the immunity can be induced.

TABLE 3
percentages of CD4 and CD8α cells of the total cell population

| Group | CD4 | CD8a |
|---|---|---|
| 1 | 12 | 8 |
| 2 | 31 | 17 |
| 3 | 35 | 21 |
| 4 | 23 | 37 |
| 5 | 25 | 36 |
| 6 | 25 | 20 |
| 7 | 25 | 29 |

### Example 2

Object of the Study

The object was to further assess the effects of a mucoadhesive adjuvant in poultry vaccination, by using next to the mucoadhesive adjuvant of Study 1, two other mucoadhesive adjuvants. The antigen was changed to inactivated IBDV to assess efficacy for another poultry antigen.

Materials and Methods

In this second study, 5 groups (N=10/group) of 3 week old SPF chickens were used. Group 1 served as a control groups which received no vaccination. At T=0, all animals of groups 2 to 5 received a primo vaccination according to the information as provided in table 4, using inactivated strain D78 of IBDV as antigen. These vaccines were applied either intramuscularly in the leg (group 2) at 0.5 ml/dose (100 units) or applied ocular/nasal (groups 3-5) at 0.2 ml/dose (5000 units). All chickens of groups 3-5 received a booster vaccination according the formulations and routes as indicated in table 4, and at the same time blood was taken for serological analysis. Group 2 received only a primo vaccination, because GNE adjuvant is known for a prolonged antigen release, not necessitating a booster vaccination. Two weeks after the booster vaccination, all chickens were exposed to a challenge infection with IBDV CS89 strain virus, which was applied via eye drop. At the same time, blood was taken for serological analysis. After challenge the chickens were monitored for clinical signs of disease or death for a period of 11 days including the challenge day.

TABLE 4
Vaccination scheme

| Group | Prime vaccination | Booster vaccination |
|---|---|---|
| 1 | — | — |
| 2 | Inac D78, 0.5 ml IM, GNE | — |
| 3 | Inac D78, 0.2 ml OC/IN, | Inac D78, 0.2 ml OC/IN, |
| | chitosan | chitosan |
| 4 | Inac D78, 0.2 ml OC/IN, | Inac D78, 0.2 ml OC/IN, |
| | alginate | alginate |
| 5 | Inac D78, 0.2 ml OC/IN, | Inac D78, 0.2 ml OC/IN, |
| | SynperonicF127 | SynperonicF127 |

Results

The serology results are indicated here below in table 5. As can be seen, the 2 log antibody titers for group 1 remained below detection level of 4.0, whereas the antibody level for the boosted groups rose to a very high level of about 10 for the IM group, as expected. However, also the groups boosted with the killed vaccine applied mucosally each showed a significant boosting effect to levels of about 7-9, corresponding to levels for adequate protection against IBDV.

TABLE 5
2log Antibody titers at booster vaccination and challenge

| Group | At booster vaccination | At challenge |
|---|---|---|
| 1 | 4.0 | 4.0 |
| 2 | 6.9 | 10.0 |
| 3 | 4.1 | 6.6 |
| 4 | 5.3 | 8.7 |
| 5 | 4.0 | 9.1 |

In table 6 the survival data after challenge are provided. As can be seen, the serology outcome corresponds to the survival data, confirming that serology corresponds to protection against the viral infection.

TABLE 6
Survival data

| Group | Survival percentage |
|---|---|
| 1 | 20% |
| 2 | 100% |
| 3 | 60% |
| 4 | 90% |
| 5 | 90% |

### Example 3

Object of the Study

The object was to further assess the effects of a mucoadhesive adjuvant in poultry vaccination, by using the two most promising mucoadhesive adjuvants of Study 2 (i.e. alginate and the polyalkylene oxide Synperonic), and changing the antigen to inactivated ARV, while at the same time using spray vaccination instead of direct OC/IN vaccination.

Materials and Methods

In total 3 groups are included in this study, using 1-week old SPF layer chickens (n=10/group). At DO all animals of groups 1-3 receive a live Reo 2177 vaccination using the commercial vaccine Nobilis Reo 2177 (MSD Animal Health) according to the information as provided in table 7. This primo vaccine is applied intramuscularly in the leg. At D28, all groups receive a booster vaccination as indicated in the table. The dose of antigen in the spray vaccines is ten times as high to account for any antigen that does not arrive at the mucosal surfaces of the chickens (but instead on the feathers, ground, walls etc.). In order to measure the efficacy by establishing the serological response as consequence of the booster vaccinations, blood is withdrawn from all animals at D28 (at booster vaccination), D42 (2 weeks after the booster vaccination) and D56 (4 weeks after the booster vaccination).

TABLE 7
Vaccination scheme

| Group | Prime vaccination | Booster vaccination |
|---|---|---|
| 1 | Nobilis Reo, 0.2 ml, IM | Inac Reo, GNE, 0.3 ml, IM |
| 2 | Nobilis Reo, 0.2 ml, IM | Inac Reo, Synperonic, 2 ml, spray |
| 3 | Nobilis Reo, 0.2 ml, IM | Inac Reo, alginate, 2 ml, spray |

Results

The serology results of the antibodies against ARV are indicated here below in table 8. As can be seen, the 2 log antibody titers for Group 1 are very high, but those of Groups 2 and 3, receiving the booster vaccination via a spray are at a corresponding level, indicating that an adequate immune response against ARV is induced. In particular the data using the mucoadhesive polyalkylene oxide provide indicate that a level of protection can be arrived at, that equals protection arrived at via IM vaccination.

TABLE 8
2log Antibody titers 2 weeks and 4 weeks
after prime and boost vaccination

| Group | At booster | 2 weeks after booster | 4 weeks after booster |
|---|---|---|---|
| 1 | 6 | 8.6 | 9.5 |
| 2 | 6.5 | 9.5 | 9.0 |
| 3 | 5 | 7.6 | 7.0 |

### Example 4

Object of the Study

The object was to further assess the effects of a mucoadhesive adjuvant in poultry vaccination, by using the alginate mucoadhesive adjuvants of Studies 2 and 3, but now in a set up to assess protection against a bacterial infection, namely Salmonella Typhimurium, in a prime-boost regimen with an inactivated multivalent Salmonella vaccine by the ocular/nasal route.

Materials and Methods

In total 2 groups are included in this study, using 40 one-day-old SPF chickens (n=20/group). At DO the animals in group 2 receive a prime vaccination with a multivalent Salmonella vaccine containing inactivated Salmonella bacteria of serovar Enteritidis, Typhimurium and Infantis and 1% alginate via the OC/IN route. At D14, the animals of group 2 receive a booster vaccination (same vaccine, same route). At D21 the animals of group 1 (control) and group 2 are challenged with virulent Salmonella typhimurium by oral gavage. At days 24, 26, 28, 31 and 35 cloacal swabs are collected from all birds. Half of the birds are euthanized of 7 days post challenge to collect spleen and liver samples as well as caecal swabs. The other half is euthanised at 14 days post challenge to collect the same.

Results

The results as a mean for all days of sample collection, are indicated here below in table 9 as a percentage of positive samples out of the total number of samples. Protection against Salmonella infection is shown.

TABLE 9
Results of Salmonella vaccination

| Samples | controls | vaccinates |
|---|---|---|
| Cloaca | 74 | 54 |
| Caecum alone | 85 | 55 |
| Spleen/liver/caecum | 73 | 52 |

1. A method of boosting an immune response in a poultry animal, which immune response is directed against a poultry pathogen, by administering a vaccine mucosally to the poultry animal, the vaccine comprising a non-live antigen of the said poultry pathogen and a mucoadhesive adjuvant.

2. The method according to claim 1, characterised in that the immune response in the poultry animal is a result of a natural infection with the poultry pathogen or the administration of a previous vaccine directed against the pathogen.

3. The method according to claim 1, characterised in that the immune response in the poultry animal is a result of a previous vaccination directed against the pathogen, by administering a previous vaccine parenterally or mucosally.

4. The method according to claim 1, characterised in that the immune response in the poultry animal is a result of a previous vaccination directed against the pathogen, by administering a previous vaccine comprising a non-live antigen of the poultry pathogen parenterally or by administering a previous vaccine comprising a live or non-live antigen of the poultry pathogen mucosally.

5. The method according to claim 2, characterised in that the vaccine comprising a non-live antigen of a poultry pathogen and a mucoadhesive adjuvant is administered within 2-6 weeks after administration of the previous vaccine.

6. The method according to claim 1, characterised in that the vaccine is administered ocular (OC) and/or intranasally (IN).

7. The method according to claim 1, characterised in that the vaccine is administered by spraying the vaccine on the poultry animal.

8. The method according to claim 1, characterised in that the mucoadhesive adjuvant has a molecular weight above 10,000 Da.

9. The method according to claim 1, characterised in that the mucoadhesive adjuvant has a molecular weight above 10,000 Da and below 1,000,000 Da.

10. The method according to claim 1, characterised in that the mucoadhesive adjuvant has a molecular weight above 10,000 Da and below 300,000 Da.

11. The method according to claim 1, characterised in that the mucoadhesive adjuvant has a pKa value below 6.

12. The method according to claim 1, characterised in that the mucoadhesive adjuvant has a pKa value between 3 and 5.

13. The method according to claim 1, characterised in that the mucoadhesive adjuvant is chosen from an alginate and a polyalkylene oxide blockcopolymer.

14. The method according to claim 1, characterised in that the mucoadhesive adjuvant is present in the vaccine in an amount between 1 and 10% w/w.

15. The method according claim 1, characterised in that the poultry pathogen is a viral pathogen.

16. The method according to claim 1, characterised in that the poultry pathogen is chosen from Newcastle Disease Virus (NDV), Avian Reo Virus (ARV), Infectious bursal disease virus (IBDV) and Salmonella typhimurium.

17. A vaccine comprising a liquid pharmaceutically acceptable carrier, a non-live antigen of a poultry pathogen and a mucoadhesive adjuvant.

18. (canceled)
